SEQUENCE LISTING (25 sequences)

SEQ ID NO: 1 - protein, 122 aa, synthetic construct
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARSGVREGRAQAWFAYWGQGTLVTVSA

SEQ ID NO: 2 - protein, 107 aa, synthetic construct
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFGGGTKLELK

SEQ ID NO: 3 - protein, 5 aa, synthetic construct
RYSVH

SEQ ID NO: 4 - protein, 16 aa, synthetic construct
MIWGGGSTDYNSALKS

SEQ ID NO: 5 - protein, 14 aa, synthetic construct
SGVREGRAQAWFAY

SEQ ID NO: 6 - protein, 11 aa, synthetic construct
KASQDVSTAVA

SEQ ID NO: 7 - protein, 7 aa, synthetic construct
SASYRYT

SEQ ID NO: 8 - protein, 9 aa, synthetic construct
QQHYSTPWT

SEQ ID NO: 9 - protein, 14 aa, synthetic construct
SGVRRGRAQAWFAY

SEQ ID NO: 10 - protein, 452 aa, synthetic construct
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARSGVRRGRAQAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 250 aa, synthetic construct
QVQLVESGGGVVQPGRSLRLSCAASGFPFRRYSVHWVRQALGKGLEWVAMIWGGGSTDYNSALKSRFTISRDTSKNTVYLKMNRLRTEDTAVYYCARSGVRRGRAQAWFAYWGKGTTVTVSSGGGGSGGGGSGGGGSDIVLTQSPGTLSLSPGERATLSCKASQDVSTAVAWYQQKPGQAPRLLIYSASYRYTGMPDRFSGSGSGTDFTLTISRLEPEDFAVYYCQQHYSTPWTFGGGTKVEIKHHHHHH

SEQ ID NO: 13 - protein, 250 aa, synthetic construct
QVQLVESGGGVVQPGRSLRLSCAASGFSLSRYSMHWVRQALGKGLEWVAVIWGGGSTYYADSVKGRFTISRDTSKNTVYLKMNRLRTEDTAVYYCARSGVRRGRAQAWFAYWGKGTTVTVSSGGGGSGGGGSGGGGSDIVLTQSPGTLSLSPGERATLSCRASQDVSTALAWYQQKPGQAPRLLIYSASTRATGMPDRFSGSGSGTDFTLTISRLEPEDFAVYYCQQHYSTPWTFGGGTKVEIKHHHHHH

SEQ ID NO: 14 - protein, 122 aa, synthetic construct
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARSGVRRGRAQAWFAYWGQGTLVTVSA

SEQ ID NO: 15 - protein, 7 aa, synthetic construct
IWGGGST

SEQ ID NO: 16 - protein, 16 aa, synthetic construct
ARSGVRRGRAQAWFAY

SEQ ID NO: 17 - protein, 6 aa, synthetic construct
QDVSTA

SEQ ID NO: 18 - protein, 107 aa, synthetic construct; identical to SEQ ID NO: 2

SEQ ID NO: 19 - protein, 9 aa, synthetic construct
QQHYSTPWT

SEQ ID NO: 20 - protein, 8 aa, synthetic construct
GFSLSRYS

SEQ ID NO: 21 - protein, 8 aa, synthetic construct
GFPFRRYS

SEQ ID NO: 22 - protein, 122 aa, synthetic construct
QVQLVESGGGVVQPGRSLRLSCAASGFPFRRYSVHWVRQALGKGLEWVAMIWGGGSTDYNSALKSRFTISRDTSKNTVYLKMNRLRTEDTAVYYCARSGVRRGRAQAWFAYWGKGTTVTVSS

SEQ ID NO: 23 - protein, 107 aa, synthetic construct
DIVLTQSPGTLSLSPGERATLSCKASQDVSTAVAWYQQKPGQAPRLLIYSASYRYTGMPDRFSGSGSGTDFTLTISRLEPEDFAVYYCQQHYSTPWTFGGGTKVEIK

SEQ ID NO: 24 - protein, 122 aa, synthetic construct
QVQLVESGGGVVQPGRSLRLSCAASGFSLSRYSMHWVRQALGKGLEWVAVIWGGGSTYYADSVKGRFTISRDTSKNTVYLKMNRLRTEDTAVYYCARSGVRRGRAQAWFAYWGKGTTVTVSS

SEQ ID NO: 25 - protein, 107 aa, synthetic construct
DIVLTQSPGTLSLSPGERATLSCRASQDVSTALAWYQQKPGQAPRLLIYSASTRATGMPDRFSGSGSGTDFTLTISRLEPEDFAVYYCQQHYSTPWTFGGGTKVEIK

(19) United States
(12) Patent Application Publication
(10) Pub. No.: US 2026/0167704 A1
(43) Pub. Date: Jun. 18, 2026

(54) MULTISPECIFIC ANTIBODIES

(71) Applicant: ABIDEA INC., Toronto (CA)

(72) Inventor: Michael Dan, Toronto (CA)

(73) Assignee: ABIDEA INC., Toronto (CA)

(21) Appl. No.: 19/389,473

(22) Filed: Nov. 14, 2025

Related U.S. Application Data
(60) Provisional application No. 63/720,541, filed on Nov. 14, 2024.

(51) Int. Cl. C07K 16/18; A61P 9/14

(52) U.S. Cl. CPC C07K 16/18; A61P 9/14; C07K 2317/622; C07K 2317/92

(57) ABSTRACT

Disclosed are two fully human single-chain Fv antibodies, IMN12 and IMN26, which specifically binds to sulfated glycosaminoglycans, wherein said antibody comprises a human heavy chain variable region sequence of SEQ ID NO: 22, or a human heavy chain variable region sequence of SEQ ID NO: 24. Antibody IMN12 show anti-glycosaminoglycan binding properties, but do not show any anti-N-glycolylneuraminic acid-binding properties, whereas antibody IMN26 exhibits both anti-glycosaminoglycan and anti-N-glycolylneuraminic acid-binding properties.

### CROSS REFERENCE TO RELATED APPLICATIONS

This application claims the benefit of U.S. Provisional Patent Application No. 63/720,541, filed Nov. 14, 2024, and entitled “MULTISPECIFIC ANTIBODIES,” the entirety of which is incorporated by reference herein.

### STATEMENT REGARDING A SEQUENCE LISTING

The present application contains a sequence listing entitled G10076944P1US.xml created Nov. 11, 2025 and is 24,605 bytes in size. The sequence listing is submitted electronically along with the filing of the present application and is hereby incorporated by reference in its entirety.

### TECHNOLOGICAL FIELD

The present disclosure relates to antibodies that inhibit the binding of LDL and remnant lipoproteins to proteoglycans. More particularly, the present disclosure relates to human antibodies and fragments thereof that bind to sulfated glycosaminoglycans (GAGs) in the extracellular matrix of the arterial endothelium, and methods of use thereof.

### BACKGROUND

The development of atherosclerosis is complex and can begin in childhood, gradually worsening with age. It often starts with damage to the arterial endothelium (the inner lining of the arteries) due to factors such as high cholesterol levels, high blood pressure, smoking, diabetes, and obesity. This damage triggers an inflammatory response that results in the accumulation of fatty plaque.

Atherosclerosis can affect any artery in the body, leading to a reduced flow of oxygenated blood to organs and tissues. Depending on the arteries involved, atherosclerosis can cause various diseases and conditions, including coronary artery disease which affects the arteries supplying blood to the heart, stroke which affects arteries leading to the brain, peripheral artery disease which affects arteries in the limbs), and kidney problems.

In the early stages, atherosclerosis is often asymptomatic and may not cause any evident symptoms until a significant blockage occurs, leading to conditions such as chest pain, leg pain, cramping, heart palpitations, shortness of breath, and mental confusion. The risk of developing atherosclerosis increases with age, and it is influenced by both modifiable lifestyle factors such as diet and physical activity, and non-modifiable factors such as genetics.

Prevention and treatment of atherosclerosis involve lifestyle changes, such as adopting a healthy diet, engaging in regular physical activity, quitting smoking, and managing weight. Medications to lower cholesterol and blood pressure may also be prescribed, and in some cases, surgical procedures, such as coronary artery bypass grafting, may be necessary to improve blood flow through affected arteries. Despite these measures, atherosclerosis cannot be completely reversed once it has developed, but its progression can be slowed, and the risk of complications can be reduced.

Low-density lipoproteins (LDL) play a critical role in the development of atherosclerosis through their interaction with sulfated GAGs and endothelial cells. This process involves several key mechanisms:

1. Transcytosis and Retention: LDL particles cross the endothelial barrier through a process called transcytosis, facilitated by receptors such as scavenger receptor class B type I (SR-BI) and activin A receptor like type 1 (ALK1). Once across the endothelium, LDL is retained in the arterial intima, where it interacts with sulfated GAGs, such as chondroitin sulfate (CS) and dermatan sulfate (DS) in the arterial wall. This interaction is mediated by specific amino acid residues on apolipoprotein B (apoB100) in LDL, which binds to negatively charged groups on sulfated GAGs (Borén, et al. 2020).

2. Oxidation of LDL: Once retained in the subendothelial space, LDL particles are susceptible to oxidative modification. Oxidized LDL (oxLDL) is highly atherogenic and can trigger a series of pathological changes. OxLDL promotes endothelial dysfunction, enhances the formation of foam cells, and contributes to the development of atherosclerotic plaques (Jiang, et al. 2022).

3. Endothelial Activation and Inflammation: OxLDL induces endothelial cells to upregulate adhesion molecules, facilitating the recruitment and adhesion of leukocytes to the endothelium. This process initiates a sterile inflammatory response, further exacerbating endothelial dysfunction and promoting the progression of atherosclerosis (Borén, et al. 2020).

4. Foam Cell Formation: Macrophages in the arterial wall uptake oxLDL via scavenger receptors, leading to cholesterol accumulation and transformation into foam cells. Foam cells are a hallmark of atherosclerotic lesions and contribute to plaque formation and instability (Borén, et al. 2020).

5. Pro-inflammatory and Immune Responses: OxLDL and the components thereof such as oxidized phospholipids, can elicit pro-inflammatory responses and activate both innate and adaptive immune pathways. This involves the recruitment of various immune cells, which perpetuate local inflammation and contribute to the chronic nature of atherosclerosis (Jiang, et al. 2022).

Overall, LDL binding to sulfated GAGs on endothelial cells, and subsequent modifications thereof, are central to the pathogenesis of atherosclerosis, driving inflammation, endothelial dysfunction, and plaque formation. These processes highlight potential therapeutic targets for preventing or slowing the progression of atherosclerosis—targets that could potentially be addressed by monoclonal antibodies (mAbs).

Broadly speaking, mAbs may be murine, human, or chimeric in nature depending on their amino acid sequence. Fully human mAbs, however, offer several advantages over murine or chimeric mAbs, primarily due to the former's compatibility with the human immune system. The following are the key benefits of fully human mAbs:

1. Reduced Immunogenicity: Fully human mAbs are less likely to be recognized as foreign by the human immune system compared to murine antibodies. This reduces the risk of immune responses such as the human anti-mouse antibody (HAMA) responses, which can lead to allergic reactions and decreased therapeutic efficacy, leading to treatment interruption or complete stoppage.

2. Improved Safety and Tolerance: Being inherently less immunogenic, fully human mAbs are generally better tolerated by human patients, including those with compromised immune systems, such as individuals undergoing chemotherapy. This improved safety profile reduces the likelihood of adverse side effects, leading to treatment adherence and completion.

3. Longer Half-life: Fully human mAbs tend to have a longer half-life in the human body than murine mAbs due to their better interaction with human Fc receptors, which play a role in recycling antibodies and extending their circulation time.

4. Enhanced Effector Functions: Fully human mAbs can efficiently engage with human immune effector mechanisms, such as complement activation and interaction with Fc receptors on immune cells. This can enhance their therapeutic efficacy in targeting diseases.

5. Ethical Considerations: The development of fully human mAbs reduces the need for animal use in antibody production, addressing ethical concerns associated with traditional methods that rely on animals.

Overall, fully human mAbs provide a more compatible and effective therapeutic option compared to murine or chimeric antibodies, particularly in terms of reduced immunogenicity and improved patient safety.

U.S. Pat. No. 8,470,322 discloses a mouse-human chimeric antibody called “Anti-SO3” that binds to sulfated GAGs. Said antibody also goes by the name chP3R99. Soto, et al. (2024) discloses that chP3R99, when acutely administered to insulin resistant rats, blocks the binding of LDL and remnant lipoproteins to the extracellular matrix of the arterial endothelium.

There is thus a need to develop fully human mAbs that bind to sulfated GAGs and that are capable of blocking the binding of LDL and remnant lipoproteins to the extracellular matrix of the arterial endothelium, thereby acutely mitigating the inflammatory pathways implicated in atherosclerotic plaque formation and progression.

### SUMMARY OF THE INVENTION

The present disclosure concerns fully human mAbs that bind to sulfated GAGs. Said mAbs can be used for blocking the binding of LDL and remnant lipoproteins to the extracellular matrix of the arterial endothelium in humans. Accordingly, the antibodies of the present disclosure can be used for mitigating the inflammatory pathways implicated in atherosclerotic plaque formation and progression.

According to a first aspect, the present invention is directed to an antibody which specifically binds to sulfated glycosaminoglycans, wherein said antibody comprises a human heavy chain variable region sequence of SEQ ID NO: 22 or 24.

According to a second aspect, the present invention is directed an antibody which specifically binds to sulfated glycosaminoglycans, wherein said antibody comprises a human light chain variable region sequence of SEQ ID NO: 23 or 25.

According to a third aspect, the present invention is directed an antibody which specifically binds to sulfated glycosaminoglycans, wherein said antibody comprises a human heavy chain variable region sequence of SEQ ID NO: 22 or 24; and a human light chain variable region sequence of SEQ ID NO: 23 or 25.

According to a fourth aspect, the present invention is directed an antibody which specifically binds to sulfated glycosaminoglycans, wherein said antibody comprises a human heavy chain variable region sequence of SEQ ID NO: 22; and a human light chain variable region sequence of SEQ ID NO: 23.

According to a fifth aspect, the present invention is directed an antibody which specifically binds to sulfated glycosaminoglycans, wherein said antibody comprises a human heavy chain variable region sequence of SEQ ID NO: 24; and a human light chain variable region sequence of SEQ ID NO: 25.

According to a sixth aspect, the present invention is directed an antibody which specifically binds to sulfated glycosaminoglycans, wherein said antibody comprises an amino acid sequence of SEQ ID NO: 12 or 13.

According to an embodiment, the antibody described herein is a monoclonal antibody.

According to an embodiment, the antibody described herein does not bind to gangliosides containing N-glycolylneuraminic acid linked α2-3 to a core galactose.

According to an embodiment, the antibody described herein binds to gangliosides containing N-glycolylneuraminic acid linked α2-3 to a core galactose.

According to an seventh aspect, the present invention is directed a single-chain Fv monoclonal antibody which specifically binds to gangliosides containing N-glycolylneuraminic acid linked α2-3 to a core galactose and/or sulfated glycosaminoglycans, wherein said single-chain Fv monoclonal antibody comprises the amino acid sequence of SEQ ID NO:12 or 13.

According to an embodiment, the single-chain Fv monoclonal antibody described herein specifically binds to sulfated glycosaminoglycans and to gangliosides containing N-glycolylneuraminic acid linked α2-3 to the core galactose.

According to an embodiment, the single-chain Fv monoclonal antibody comprises the amino acid sequence of SEQ ID NO:13.

According to an embodiment, the single-chain Fv monoclonal antibody described herein does not bind to gangliosides containing N-glycolylneuraminic acid linked α2-3 to the core galactose.

According to an embodiment, the single-chain Fv monoclonal antibody comprises the amino acid sequence of SEQ ID NO:12.

According to a eighth aspect, the present invention is directed a monoclonal antibody which specifically binds to gangliosides containing N-glycolylneuraminic acid linked α2-3 to a core galactose and/or sulfated glycosaminoglycans, wherein said monoclonal antibody comprises a heavy chain variable region comprising the amino acid sequence of SEQ ID NO: 22 or 24 and a light chain variable region comprising the amino acid sequence of SEQ ID NO: 23 or 25.

According to an embodiment, the monoclonal antibody specifically binds to sulfated glycosaminoglycans and to gangliosides containing N-glycolylneuraminic acid linked α2-3 to the core galactose.

According to an embodiment, the monoclonal antibody comprises the amino acid sequence of SEQ ID NO:13.

According to a ninth aspect, the present invention is directed a method of reducing and/or preventing binding of low density lipoprotein (LDL) to the extracellular matrix of the arterial endothelium in a subject in need thereof, the method comprising administering the antibody described herein to the subject.

According to an tenth aspect, the present invention is directed a method of reducing and/or preventing binding of low density lipoprotein (LDL) blood vessels in a subject in need thereof, the method comprising administering the antibody described herein to the subject.

According to an embodiment, the method is for reducing and/or preventing binding of LDL to the endothelial surface of blood vessels.

According to an embodiment, the blood vessels comprise atherosclerotic arteries.

According to an eleventh aspect, the present invention is directed a method of diagnosing atherosclerosis in a subject, the method comprising administering the antibody described herein to the subject, and visualizing the antibody in the subject.

According to a twelfth aspect, the present invention is directed to a method of imaging atherosclerosis in a subject, the method comprising administering the antibody of described herein to the subject, and visualizing the antibody in the subject.

According to an embodiment, the antibody comprises a tag for visualising and localizing the antibody in the subject.

According to an embodiment, the subject is a mammal.

According to an embodiment, the mammal is a human.

### BRIEF DESCRIPTION OF THE DRAWINGS

Other and further aspects and advantages of the present invention will be better understood upon the reading of the illustrative embodiments about to be described or will be indicated in the appended claims, and various advantages not referred to herein will occur to one skilled in the art upon employment of the invention in practice. Having thus generally described the nature of the invention, reference will now be made to the accompanying drawings, showing by way of illustration, embodiments thereof, and in which:

FIG. 1 shows images of SDS-PAGE gels of: A) antibody IMN12 (94.2% purity), B) antibody IMN26 (94.0% purity), and C) attempted grafting of the Chothia CDRs of antibody chP3R99 into a fully human scFv framework. See text for details on the construction of antibodies IMN12 and IMN26.

FIG. 2 shows a schematic of the following GAG structures used in the microarrays: hyaluronic acid, heparin, chondroitin sulfate AC, and chondroitin sulfate D.

FIG. 3 shows a schematic of the following GAG structures used in the microarrays: dermatan sulfate, heparan sulfate (low and intermediate sulfation), heparan sulfate (high sulfation), and keratan sulfate.

FIG. 4 shows the GAG binding profile of chP3 at a concentration of 50 μg/mL (top) and 10 μg/mL (bottom), RFU: relative fluorescent unit.

FIG. 5 shows the GAG binding profile of IMN12 at a concentration of 2 μg/mL (top) and 0.4 μg/mL (bottom), RFU: relative fluorescent unit.

FIG. 6 shows the GAG binding profile of chP3R99 at a concentration of 2 μg/mL (top) and 0.4 μg/mL (bottom), RFU: relative fluorescent unit.

FIG. 7 shows the GAG binding profile of IMN26 at a concentration of 2 μg/mL (top) and 0.4 μg/mL (bottom), RFU: relative fluorescent unit.

FIG. 8 shows the GSL glycan binding profile of chP3 at a concentration of 50 μg/mL (top) and 10 μg/mL (bottom), RFU: relative fluorescent unit.

FIG. 9 shows the GSL glycan binding profile of IMN12 at a concentration of 10 μg/mL (top) and 2 μg/mL (bottom), RFU: relative fluorescent unit.

FIG. 10 shows the GSL glycan binding profile of chP3R99 at a concentration of 2 μg/mL (top) and 0.4 μg/mL (bottom), RFU: relative fluorescent unit.

FIG. 11 shows the GSL glycan binding profile of IMN26 at a concentration of 2 μg/mL (top) and 0.4 μg/mL (bottom), RFU: relative fluorescent unit.

### DETAILED DESCRIPTION

Fully human mAbs that bind to sulfated GAGs will be described hereinafter. Although the invention is described in terms of specific illustrative embodiments, it is to be understood that the embodiments described herein are by way of example only and that the scope of the invention is not intended to be limited thereby.

The terminology used herein is in accordance with definitions set out below.

By “about”, it is meant that the value can vary within a certain range depending on the margin of error of the method or device used to evaluate or measure. A margin of error of 10% is generally accepted.

As used herein, the expression “associated with”, when used in the context of a glycosaminoglycan and a protein core, signifies that the glycosaminoglycan is covalently bound to the protein core to form a proteoglycan. For example, “heparan sulfate in association with Syndecan-1” means that the heparan sulfate is covalently bound to Syndecan-1.

“High sulfation glycosaminoglycan” means a glycosaminoglycan with greater than two sulfate groups per disaccharide unit. For the sake of clarity, chondroitin-4-sulfate (also called chondroitin sulfate A) is not a high sulfation glycosaminoglycan.

“Low sulfation heparan sulfate” means an heparan sulfate with less than one sulfate groups per disaccharide unit.

“Intermediate sulfation heparan sulfate” means an heparan sulfate with about two sulfate groups per disaccharide unit.

“Isolated heparan sulfate” refers to either a heparan sulfate molecule that is not attached to a protein core, or one that has been bound to a glycan microarray for detection purposes. Glycan microarrays comprise glycan molecules with amino linkers that have been printed onto N-hydroxysuccinimide (NHS)-activated glass microscope slides via covalent amide linkages.

“GSL glycan” refers to the carbohydrate component of a glycosphingolipid (GSL). GSL glycans may contain a sialic acid, such as N-acetylneuraminic acid or N-glycolylneuraminic acid.

An “anti-GAG antibody” in the context of the present disclosure is an antibody that specifically binds to an isolated glycosaminoglycan (GAG), as opposed to an antibody that only binds to a GAG that itself is covalently bound to a core protein.

The following abbreviations are used throughout the present disclosure are defined hereinbelow for convenience:
Ab: antibody
ABMT: autologous bone marrow transplantation
Ag: antigen
CDR: complementarity-determining regions
CS: chondroitin sulfate
CSD: chondroitin-2,6-sulfate, or chondroitin sulfate D
CSE: chondroitin-4,6-sulfate, or chondroitin sulfate E
dp: degree of polymerization
DS: dermatan sulfate
EC: endothelial cell
ELISA: enzyme-linked immunosorbent assay
FACS: fluorescence activated cell sorting
Fc: fragment crystallizable
FR: framework regions
HA: hyaluronic acid
Hep: heparin
hFc: human fragment crystallizable
HS: heparan sulfate
Hsp: heat shock protein
Ig: immunoglobulin
IgG: immunoglobulin G
IMGT: International Immunogenetics Information System™
KS: keratan sulfate
mAb: monoclonal antibody
Neu5Gc: N-glycolylneuraminic acid
NeuGcGM3: N-glycolylneuraminic GM3 ganglioside
PG: proteoglycan
scFv: single-chain variable fragment
V_{H}: variable region heavy chain
V_{L}: variable region light chain

Recent studies have highlighted the role of immune responses, particularly the involvement of T cells, B cells, and various cytokines, in the formation and progression of atherosclerotic plaques. Immunotherapy strategies are being explored to alleviate atherosclerosis by inducing a protective immune response through certain auto-antigens or exogenous antigens. Clinical trials have demonstrated the association of atherosclerosis with the presence of immune cells and immune factors in the body, suggesting that immunotherapy could significantly impact the management of atherosclerosis (Meng, et al., 2022).

Immunotherapeutic approaches that are being explored include targeting specific inflammatory pathways implicated in atherosclerotic plaque formation. For instance, therapies directed at pro-inflammatory cytokines such as IL-1β, IL-6, TNFα, and CCL2 have shown promise in slowing the progression of atherosclerosis in animal models and improving cardiovascular outcomes (Khambhati, et al, 2018). The mAb canakinumab, which targets IL-1β, has been associated with reduced risk of adverse cardiovascular events in a randomized, placebo-controlled trial (Shah, et al. 2018).

Despite these advances, many challenges remain in translating immunotherapy from animal studies to clinical applications. These challenges include ensuring the safety and stability of vaccines, determining the effective duration of immunization, identifying the effective cut-off point of clinical trials, and assessing the impact on existing atherosclerosis therapeutic measures.

An antibody, also known as an immunoglobulin (Ig), is a protective protein produced by the immune system in response to the presence of antigens, which are foreign substances that enter the body. Antigens can include a wide range of substances such as bacteria, viruses, fungi, parasites, and toxic chemicals. Antibodies play a crucial role in the body's defense mechanism by identifying and neutralizing these antigens.

Structurally, antibodies are Y-shaped molecules composed of four polypeptide chains: two identical heavy chains and two identical light chains, connected by disulfide bonds. The tips of the “Y” shape contain regions known as paratopes that are specific to particular antigens, allowing antibodies to bind with high precision to epitopes (specific parts) on the antigens. Each antibody heavy or light chain includes a variable region and constant region. And each variable region is comprised of 4 framework sequences that are interspersed with 3 CDR sequences (also called hypervariable regions). The 6 CDR sequences (3 on the heavy chain and 3 on the light chain) are primarily responsible for determining antibody specificity; however, framework sequences may also occasionally participate in the binding process. Each heavy or light chain variable region may be numbered according to the Kabat, Chothia, or IMGT numbering system (vide infra).

Antibodies are produced by specialized white blood cells called B lymphocytes (or B cells). When an antigen binds to the B cell surface, it stimulates the B cell to divide and mature into a group of identical cells known as a clone. These mature B cells, called plasma cells, secrete millions of antibodies into the bloodstream and lymphatic system. The production of antibodies continues until all antigen molecules are removed, and antibodies can remain in circulation for several months, providing extended immunity against that particular antigen.

There are five main classes of antibodies in humans, each with distinct functions and properties: IgA, IgD, IgE, IgG, and IgM. These classes are differentiated based on the structure of their heavy chains and their roles in the immune response. For example, IgG is the most abundant antibody in blood and tissue fluids and is the only class capable of crossing the placenta to provide immunity to the fetus. IgM is usually the first antibody produced in response to an antigen and is effective at agglutinating microbes. IgA is found in many body secretions, such as tears and saliva, providing protection at mucosal surfaces. IgE is involved in allergic reactions by binding to mast cells and basophils. The function of IgD is less understood but is believed to play a role in the early stages of the immune response.

Antibodies have a wide range of applications in medicine and research, including the diagnosis of diseases, treatment of certain conditions through the use of mAbs, and the development of vaccines to elicit an immune response.

The Kabat, Chothia, and IMGT numbering systems are three distinct methods developed to standardize the numbering of amino acid residues in antibody variable regions. These systems facilitate the comparison of sequences and structures across different antibodies by providing a consistent framework for identifying complementarity-determining regions (CDRs) and framework regions (FRs). Each system has its unique approach and has been developed to address specific needs within the field of immunology and antibody research.

The Kabat numbering system, introduced by Wu and Kabat in 1970, is based on the alignment of amino acid sequences of antibody variable regions. It was the first scheme proposed and relies solely on sequence data. This system identifies specific positions where insertions and gaps may occur in CDRs and FRs, with additional amino acid insertions annotated with letters (e.g., 100A, 100B, etc.). However, the Kabat system has limitations, particularly in handling antibodies of unconventional length or with unconventional insertions or deletions, as it was developed from a limited dataset and without considering the three-dimensional structure of antibodies.

The Chothia numbering system, introduced by Chothia in 1987, incorporates structural information into the numbering scheme. It is similar to the Kabat system but modifies the definition by aligning the variable region crystal structures to form CDRs, rather than relying solely on sequence-based alignment. This structure-based approach allows for differences in amino acid insertion points, especially for VLCDR1 and VHCDR1, and aims to ensure that the numbering corresponds with the three-dimensional structures of antibodies of typical length. The Chothia system places insertions in VLCDR1 and VHCDR1 at structurally correct positions, unlike the Kabat scheme.

The IMGT numbering system, defined by Marie-Paule Lefranc, is designed to easily compare sequences of immunoglobulins (IG) and T cell receptors (TR) from all vertebrate species, as well as sequences with an immunoglobulin-like domain. It avoids insertion codes (such as 27A, 27B, etc.) used in other systems and instead defines the longest antibody sequence possible, removing residues as necessary. The IMGT system provides a standardized delimitation of the framework regions and CDRs, ensuring that conserved amino acids always have the same position regardless of the IG or TR chain type, domain (variable or constant), or species. This system is based on a complete reference gene database, making it widely applicable and used.

The concept of the idiotypic network suggests that the immune system is regulated through a complex interaction of antibodies and lymphocytes. Immunization with an antigen leads to the production of antibodies (Ab1) against this antigen. These Ab1 antibodies can then stimulate the production of anti-idiotypic antibodies (Ab2), some of which can mimic the three-dimensional structure of the original antigen (referred to as Ab2 β). When used as immunogens, these Ab2 β antibodies can induce the production of anti-anti-idiotypic antibodies (Ab3) that have specificity similar to an Ab1.

The present disclosure describes design improvements to an anti-GAG antibody called chP3R99. When chP3R99 is administered acutely, it prevents the binding of LDL and remnant lipoproteins to the extracellular matrix of the arterial endothelium, including the endothelium of atherosclerotic arteries (Soto, et al. 2024). Chronic administration of antibody chP3R99 has been shown to activate the anti-Id network of mice, rabbits, and rats, thereby leading to the formation of Ab3 antibodies that also bind to sulfated GAGs (specifically Hep, CS, and DS) (Brito, et al. 2012; Soto, et al. 2012; Delgado-Roche, et al. 2013).

The Multispecific Murine mAb, P3, and Initial Improvements Thereof

Vázquez, et al. (1995) discloses a murine IgM monoclonal antibody (mAb) called P3, generated by immunizing BALB/c mice with GcGM3, a Neu5Gc-containing ganglioside that functions in humans as a cancer-specific biomarker (Labrada, et al. 2018; Dhar, et al. 2019). Initial characterization of P3 indicated that it binds to GcGM3 (as expected) as well as to ganglioside GcGM2, both of which contain Neu5Gc. No binding was observed to gangliosides that contain N-acetylneuraminic acid (NANA). Unexpectedly, P3 was also observed to bind to the sulfated gangliosides IV³SO₃-Gg₄Cer (sulfo-asialo-GM1) and I³SO₃-GalCer (SM4), both of which contain a sulfate group linked to a galactose. Thus, from the outset, P3 exhibited the properties of a multispecific antibody.

The nature of the interaction between P3 and its epitope is primarily Coulombic. Vázquez, et al. (1995) noted that “P3 MAb recognition seems to require a net negative charge on the glycolipids, either in the form of the carboxyl group of sialic acid, or a sulfate group in sulfatides. In addition, binding to sialic acid requires the effective charge of the N-glycolyl function. Thus, electrostatic interactions might support the unique high specificity of the P3 MAb.”

Further studies by Moreno, et al. (1998) indicated that P3 reacts with the gangliosides GcGM2, GcGM3, GcGD3, GcGcGD3, and AcGcGD3 but not GcAcGD3. This makes P3 unique in that it recognizes Neu5Gc on several different types of sugar chain. Molecular modeling was used to analyze the binding data, including the finding that P3 selectively recognizes the internal Neu5Gc in GD3. The authors concluded that “ . . . sialic acid binds the P3 antibody through its upper face (the one on which the carboxyl group is exposed) and the C4-C5 side of the sugar ring, where none or very little contact between the galactose residue and the protein is evident. Conformational analysis of GD3 revealed that, despite the large flexibility of the NeuGca8NeuGc linkage, the P3 binding epitope on the external sialic acid is not well exposed for any of the possible conformations this linkage can adopt, whereas the internal sialic acid presents the epitope in a proper way for several of these conformations.” It was thus determined that antibody P3 binds to Neu5Gc linked α2-3 to a core galactose, and that the presence of NANA on a particular ganglioside does not preclude P3 from binding thereto (e.g., AcGcGD3) so long as there's a Neu5Gc linked α2-3 to the core galactose of said ganglioside.

Perez, et al. (2001) discloses the P3 variable region heavy chain sequence (defined in the present disclosure as SEQ ID NO:1) and the P3 variable region light chain sequence (defined in the present disclosure as SEQ ID NO:2). The authors also disclose the P3 V_{H} and V_{L} CDRs according to the Kabat numbering system. These are defined in the present disclosure as SEQ ID NOs: 3-8.

López-Requena, et al. (2003) discloses that in a syngeneic model, P3 is able to elicit a strong anti-idiotypic (Ab2) antibody response, even in the absence of adjuvant or carrier proteins. Thus, P3 functions as an anti-idiotypic vaccine in BALB/c mice. The authors also disclose the construction of a human IgG1 chimeric version of P3, so-called chP3, which retained the binding specificity of P3. Upon immunization of BALB/c mice with chP3, the authors were able to demonstrate immunodominance of the variable region. The anti-idiotypic response was strong and in most of the mice was significantly higher than the anti-isotypic response, even though 70% of the chimeric molecule is xenogeneic with respect to the animal model.

Thus, chP3 represents the first in a series of design improvements in the murine mAb, P3. With each subsequent improvement, P3 was to become more compatible for use in humans.

López-Requena, et al. (2007) describes the construction of four mutants of the P3 antibody, where arginine residues in the heavy chain CDRs were substituted by serine residues, and the mutants' interactions with a P3 anti-idiotype mAb called 1E10 as well as GcGM3 ganglioside was evaluated. The authors also investigated the mutants' immunogenic properties in BALB/c mice. They concluded that the P3 VHCDR1 R₃₁ residue appears to play a central role in reactivity and antigenicity, whereas the P3 VHCDR3 R₁₀₀ₐ residue seems to be more involved in immunogenicity.

The second significant design improvement in the murine mAb, P3, is disclosed in WO 2010/127642, which was filed on May 4, 2009, and which describes an anti-SO₃ chimeric antibody with heavy and light chain variable regions that are identical to P3 except for the substitution of arginine for glutamic acid in position 99 of the heavy chain variable region. The resulting VHCDR3, which is defined in the present disclosure as SEQ ID NO:9, contains an ideal Cardin-Weintraub motif (Cardin and Weintraub, 1989), which helps to explain the antibody's affinity for GAGs. WO 2010/127642 also discloses that the anti-SO₃ chimeric antibody binds to bovine brain sulfatides, heparin (Hep), macrophages, and atherosclerotic plaque in human aortas. The anti-SO₃ chimeric antibody of WO 2010/127642 also induced an anti-Hep immune response in mice immunized with 50 μg of the anti-SO₃ chimeric antibody injected subcutaneously every 2 weeks for a total of 4 doses. The anti-SO₃ chimeric antibody also showed an anti-atherosclerotic effect in rabbits in a Lipofundin-induced atherosclerosis model.

Thus, the twice improved version of P3 (as disclosed in WO 2010/127642) is a 70% human, multispecific antibody that binds to Neu5Gc-containing gangliosides, sulfatides, and Hep (which itself is a sulfated GAG). Said chimeric antibody has a clinical effect in a rabbit model of atherosclerosis and it binds to human atherosclerotic plaques; all of which make it a worthwhile candidate for further improvement.

Fernández-Marrero, et al. (2011) adds further detail regarding the impact of substituting arginine for glutamic acid in position 99 of the heavy chain variable region of P3. The authors state that the P3 E₉₉→R mutant exhibited increased reactivity with GcGM3 relative to the wild type P3, as well as a cytotoxic effect on ganglioside-expressing L1210 tumor cells previously unseen with the wild type P3.

Brito, et al. (2012) discloses that the anti-SO₃ chimeric antibody of WO 2010/127642, now called chP3R99, reduced atherosclerosis in apolipoprotein E-deficient mice fed a high fat, high cholesterol (HFHC) diet. The anti-atherosclerotic effect was associated with increased mice sera reactivity against Hep and sulfated GAGs, including CS and DS. In addition, purified IgG from chP3R99-immunized mice blocked the retention of apolipoprotein E-containing lipoproteins within the arterial wall of apolipoprotein E^{-/-} mice. The authors concluded that “ . . . the atheroprotective effect [of chP3R99] likely involves the induction of anti-anti-idiotypic antibodies capable of blocking the interaction between intimal proteoglycans and LDL, thereby inhibiting LDL retention into the arterial wall.”

Soto, et al. (2012) discloses that chP3R99 not only reduced atherosclerotic lesions, but also prevented their formation. Subcutaneous immunization of New Zealand White rabbits with chP3R99 (100 μg, 3 doses at weekly intervals) prevented Lipofundin-induced atherosclerosis with a 22-fold reduction in the intima:media ratio (p<0.01). Additionally, immunization with chP3R99 suppressed macrophage infiltration in the aorta and preserved redox status. The atheroprotective effect was associated with the induction of anti-CS antibodies in chP3R99-immunized rabbits, capable of blocking CS-LDL binding and LDL oxidation. Also disclosed by Soto, et al. (2012) is that chP3R99 binds strongly to Hep/HS, CS, and DS in an ELISA assay, whereas reactivity with hyaluronic acid (HA) was not as strong. Finally, the authors disclose the third significant design improvement of P3: a variant of chP3R99 called chP3R99-LALA, which features impaired FcγR and complement binding.

U.S. Pat. No. 8,470,322 was issued on Jun. 25, 2013, and claims a mAb which specifically binds to sulfatides and sulfated proteoglycans, said mAb having all 6 Kabat CDRs of antibody chP3R99 (see claim 1). U.S. Pat. No. 8,470,322 neither claims an antibody that binds to Neu5Gc-containing gangliosides (even though the antibody it discloses does so). Moreover, U.S. Pat. No. 8,470,322 is completely silent with respect to antibodies that bind to Neu5Gc-containing gangliosides. Furthermore, U.S. Pat. No. 8,470,322 does not provide any examples of an antibody that binds to sulfated proteoglycans (even though it claims that the antibody it discloses does so). U.S. Pat. No. 8,470,322 only features an example of antibody binding to Hep, but Hep does not form sulfated proteoglycans.

Soto, et al. (2013) discloses that ^{99m}Tc-labeled chP3R99 preferentially accumulated in arterial atherosclerotic lesions, which “supports the potential use of this anti-glycosaminoglycans antibody for diagnosis and treatment of atherosclerosis.”

Delgado-Roche, et al. (2015) discloses that therapeutic immunizations with chP3R99-LALA halted atherosclerotic lesion progression in apolipoprotein E-deficient mice fed with a HFHC diet. Sarduy, et al. (2017) discloses that chP3R99-LALA reduced atherosclerotic lesions to a similar extent in both young male and female apolipoprotein E-deficient mice fed a HFHC diet, with a dose of 200 vs. 50 μg resulting in a more striking reduction of aortic atherosclerotic lesions. And Brito, et al. (2017) discloses that when apolipoprotein E-deficient mice were fed with a HFHC diet, then switched to a regular chow diet and immunized with chP3R99, atherosclerotic lesions which had previously developed, regressed in a significant number of mice.

Finally, Soto, et al. (2024) discloses that both acute and long-term administration of chP3R99 reduced LDL and remnant lipoprotein interaction with proteoglycans in insulin-resistant rats. A five-week vaccination study with chP3R99 (200 μg s.c., once a week) reduced arterial lipoprotein retention, and was associated with the production of anti-CS antibodies (Ab3) able to accumulate in the arteries.

Further Design Improvements to the Murine mAb, P3

Improving a murine mAb for use in humans involves a process known as humanization, which is designed to reduce the immunogenicity of the antibody when administered to humans and to enhance its therapeutic efficacy. The goal is to modify the murine antibody so that the human immune system does not recognize it as foreign, thereby reducing the risk of adverse immune responses while retaining the antibody's specific binding affinity to its target antigen. Several strategies have been developed for this purpose:

Creating chimeric antibodies is an initial step towards humanization. This involves replacing the constant regions of a murine antibody with those of a human antibody while retaining the murine variable regions that determine antigen specificity. Although chimeric antibodies (e.g., chP3R99) are less immunogenic than fully murine antibodies (e.g., P3), they can still elicit an immune response due to the murine variable regions.

Complementarity-determining region (CDR) grafting is a more refined approach to humanization. It involves transplanting the CDRs of a murine antibody (the parts that bind to the antigen) into a human antibody framework. This method aims to maintain the antigen-binding specificity of the original murine antibody while significantly reducing its immunogenicity by using a predominantly human antibody structure.

Specificity-determining residue (SDR) grafting is a variation of CDR grafting that involves transferring only the key residues within the CDRs that are critical for antigen binding. This method can potentially reduce immunogenicity further by minimizing the number of murine residues introduced into the human antibody framework.

Resurfacing involves modifying the surface of the variable regions of a murine antibody to resemble that of a human antibody. This is achieved by identifying and replacing murine residues that are exposed to the immune system with their human counterparts, while retaining the internal structure that is crucial for antigen binding.

This method involves creating a library of humanized antibodies displayed on the surface of mammalian cells. High-affinity binders are then selected through bio-panning or fluorescence-activated cell sorting (FACS). This approach allows for the selection of humanized antibodies in a full-size IgG format that retain or increase the original affinity of the mouse antibody.

Recent advancements include the use of machine learning to predict and design humanized antibodies. Algorithms can analyze large datasets of antibody sequences to identify patterns and suggest modifications that reduce immunogenicity while preserving or enhancing binding affinity,

In the present disclosure, antibody chP3R99 was further improved by means of CDR grafting onto a human variable region framework. The human heavy chain variable region that was employed is disclosed as SEQ ID NO:2 in WO 97/44461, and the human light chain variable region that was employed is disclosed as SEQ ID NO:5 in WO 97/44461.

The 6 Kabat CDRs of chP3R99 were first grafted onto a human variable region framework. The resulting construct (IMN12) failed to replicate the anti-Neu5Gc-binding properties of chP3R99 even though it retained the anti-GAG binding properties. This was an entirely unexpected result, since U.S. Pat. No. 8,470,322 effectively claims that any mAb having the 6 Kabat CDRs of chP3R99 would be functionally equivalent to chP3R99. The results of the present disclosure do not support this claim, therefore, U.S. Pat. No. 8,470,322 does not fully teach the antibody of the present disclosure.

Equally unexpected was the finding that an antibody comprised of the 6 IMGT CDRs of chP3R99, grafted onto a human variable region framework (i.e., IMN26), would retain both the anti-Neu5Gc and the anti-GAG binding properties of chP3R99. These findings were unexpected as such an antibody was not contemplated by U.S. Pat. No. 8,470,322 even though functionally, said antibody is the equivalent of chP3R99.

Having thus introduced the field of endeavour of the present disclosure, the present invention will be more readily understood by describing specific embodiments.

According to a first aspect, the present invention is directed to an antibody which specifically binds to sulfated glycosaminoglycans, wherein said antibody comprises a human heavy chain variable region sequence of SEQ ID NO: 22 or 24.

According to a second aspect, the present invention is directed an antibody which specifically binds to sulfated glycosaminoglycans, wherein said antibody comprises a human light chain variable region sequence of SEQ ID NO: 23 or 25.

According to a third aspect, the present invention is directed an antibody which specifically binds to sulfated glycosaminoglycans, wherein said antibody comprises a human heavy chain variable region sequence of SEQ ID NO: 22 or 24; and a human light chain variable region sequence of SEQ ID NO: 23 or 25.

According to a fourth aspect, the present invention is directed an antibody which specifically binds to sulfated glycosaminoglycans, wherein said antibody comprises a human heavy chain variable region sequence of SEQ ID NO: 22; and a human light chain variable region sequence of SEQ ID NO: 23.

According to a fifth aspect, the present invention is directed an antibody which specifically binds to sulfated glycosaminoglycans, wherein said antibody comprises a human heavy chain variable region sequence of SEQ ID NO: 24; and a human light chain variable region sequence of SEQ ID NO: 25.

According to a sixth aspect, the present invention is directed an antibody which specifically binds to sulfated glycosaminoglycans, wherein said antibody comprises an amino acid sequence of SEQ ID NO: 12 or 13.

According to an embodiment, the antibody is a monoclonal antibody.

According to an embodiment, the antibody does not bind to gangliosides containing N-glycolylneuraminic acid linked α2-3 to a core galactose.

According to an embodiment, the antibody binds to gangliosides containing N-glycolylneuraminic acid linked α2-3 to a core galactose.

According to a seventh aspect, the present invention is directed a single-chain Fv monoclonal antibody which specifically binds to gangliosides containing N-glycolylneuraminic acid linked α2-3 to a core galactose and/or sulfated glycosaminoglycans, wherein said single-chain Fv monoclonal antibody comprises the amino acid sequence of SEQ ID NO:12 or 13.

According to an embodiment, the single-chain Fv monoclonal antibody specifically binds to sulfated glycosaminoglycans and to gangliosides containing N-glycolylneuraminic acid linked α2-3 to the core galactose.

According to an embodiment, the single-chain Fv monoclonal antibody comprises the amino acid sequence of SEQ ID NO:13.

According to an embodiment, the single-chain Fv monoclonal antibody does not bind to gangliosides containing N-glycolylneuraminic acid linked α2-3 to the core galactose.

According to an embodiment, the single-chain Fv monoclonal antibody comprises the amino acid sequence of SEQ ID NO:12.

According to an eighth aspect, the present invention is directed a monoclonal antibody which specifically binds to gangliosides containing N-glycolylneuraminic acid linked α2-3 to a core galactose and/or sulfated glycosaminoglycans, wherein said monoclonal antibody comprises a heavy chain variable region comprising the amino acid sequence of SEQ ID NO: 22 or 24 and a light chain variable region comprising the amino acid sequence of SEQ ID NO: 23 or 25.

According to an embodiment, the monoclonal antibody specifically binds to sulfated glycosaminoglycans and to gangliosides containing N-glycolylneuraminic acid linked α2-3 to the core galactose.

According to an embodiment, the monoclonal antibody comprises the amino acid sequence of SEQ ID NO:13.

According to a ninth aspect, the present invention is directed a method of reducing and/or preventing binding of low density lipoprotein (LDL) to the extracellular matrix of the arterial endothelium in a subject in need thereof, the method comprising administering the antibody of described herein to the subject.

According to a tenth aspect, the present invention is directed a method of reducing and/or preventing binding of low density lipoprotein (LDL) blood vessels in a subject in need thereof, the method comprising administering the antibody described herein to the subject.

According to an embodiment, the method is for reducing and/or preventing binding of LDL to the endothelial surface of blood vessels.

According to an embodiment, the blood vessels comprise atherosclerotic arteries.

According to an eleventh aspect, the present invention is directed a method of diagnosing atherosclerosis in a subject, the method comprising administering the antibody described herein to the subject, and visualizing the antibody in the subject.

According to a twelfth aspect, the present invention is directed to a method of imaging atherosclerosis in a subject, the method comprising administering the antibody of described herein to the subject, and visualizing the antibody in the subject.

According to an embodiment, the antibody comprises a tag for visualising and localizing the antibody in the subject.

According to an embodiment, the subject is a mammal.

According to an embodiment, the mammal is a human.

The present invention will be more readily understood by referring to the following examples which are given to illustrate the invention rather than to limit its scope.

### EXAMPLES

### Example 1: Production of IMN12 and IMN26

Antibody chP3 was commercially sourced from Creative Biolabs, Inc. (Shirley, NY)(Product no. PABL-663). Antibody chP3R99 was custom produced by Sino Biological US, Inc. (Wayne, PA) as an IgG1 according to the V_{H} and V_{L} sequences disclosed in Perez, et al. (2001), with the glutamate to arginine substitution at Kabat position 99 of the V_{H} sequence as disclosed in Fernández-Marrero, et al. (2011). The heavy chain of chP3R99 is given as SEQ ID NO:10 and the light chain of chP3R99 is given as SEQ ID NO:11. Antibodies IMN12 and IMN26 were custom produced by Sino Biological US, Inc. as scFvs with a V_{H}→linker→V_{L}→polyHis tail configuration (SEQ ID NO:12 and SEQ ID NO:13, respectively).

For IMN12 and IMN26, the target genes were amplified by PCR and inserted into the corresponding expression vector. The sequence of the constructed vector was validated by sequencing. The correct plasmid was amplified and transmitted to downstream. For transient transfection, the plasmids were mixed with transfection reagents at an optimal ratio and then added into the flask or the bioreactor containing HEK293. The cells were cultured in a serum-free medium and maintained in Erlenmeyer Flasks on an orbital shaker or the bioreactor by a suitable stirring speed at 37° C. for 6 days. The cell culture broth was centrifuged, cell culture supernatant was loaded onto affinity purification column at an appropriate flow rate, and the purified protein was analyzed by SDS-PAGE. The purity of the final product was >94%.

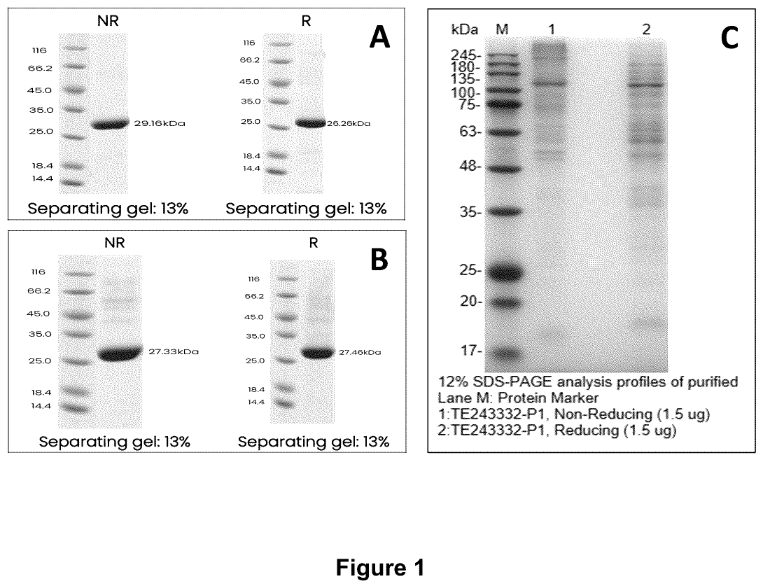

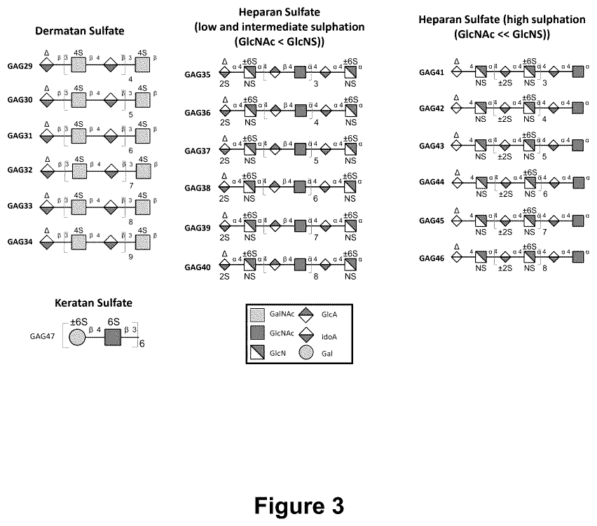

The P3 heavy chain variable region sequence and corresponding Kabat CDRs are disclosed in FIG. 1 of Perez, et al. (2001). The P3 light chain variable region sequence and corresponding Kabat CDRs are disclosed in FIG. 3 of Perez, et al. (2001).

Antibody IMN12 (SEQ ID NO:12) is a scFv with the 6 Kabat CDRs of chP3R99 (SEQ ID NOS: 3, 4, 6-9) grafted onto a human variable region framework.

The following tables offers a comparison of the Kabat CDRs of chP3R99, IMN12, and IMN26:

| | VHCDR1 | VHCDR2 | VHCDR3 |
|---|---|---|---|
| chP3R99 | RYSVH | MIWGGGST | SGVRRGR |
| | | DYNSALKS | AQAWFAY |
| IMN12 | RYSVH | MIWGGGST | SGVRRGR |
| | | DYNSALKS | AQAWFAY |
| IMN26 | RYSMH | VIWGGGST | SGVRRGR |
| | | YYADSVKG | AQAWFAY |
| | VLCDR1 | VLCDR2 | VLCDR3 |
| chP3R99 | KASQDV | SASYRYT | QQHYSTPWT |
| | STAVA | | |
| IMN12 | KASQDV | SASYRYT | QQHYSTPWT |
| | STAVA | | |
| IMN26 | RASQDV | SASTRAT | QQHYSTPWT |
| | STALA | | |

The following tables offers a comparison of the IHGT CDRs of chP3R99, IMN12, and IMN26:

| | VHCDR1 | VHCDR2 | VHCDR3 |
|---|---|---|---|
| chP3R99 | GFSLSRYS | IWGGGST | ARSGVRRG |
| | | | RAQAWFAY |
| IMN12 | GFPFRRYS | IWGGGST | ARSGVRRG |
| | | | RAQAWFAY |
| IMN26 | GFSLSRYS | IWGGGST | ARSGVRRG |
| | | | RAQAWFAY |
| | VLCDR1 | VLCDR2 | VLCDR3 |
| chP3R99 | QDVSTA | SAS | QQHYSTPWT |
| IMN12 | QDVSTA | SAS | QQHYSTPWT |
| IMN26 | QDVSTA | SAS | QQHYSTPWT |

Attempts by Creative Biolabs Inc. at producing a scFv comprised of the 6 Chothia CDRs of chP3R99 grafted onto a human variable region framework, whether as a V_{H}→V_{L} or as a V_{H}→V_{L} orientation, were unsuccessful as evidenced by the appearance of multiple abnormal bands on an SDS-PAGE gel (see FIG. 1C).

CDR grafting, which involves transferring the complementarity-determining regions (CDRs) from a non-human antibody to a human antibody framework, does not always result in an antibody that can be easily expressed, or that retains its original function. Several factors contribute to this variability:

1. Framework Compatibility: The success of CDR grafting depends significantly on the compatibility between the CDRs and the human framework onto which they are grafted. In some cases, the human framework may not support the correct conformation of the CDRs, leading to a loss of binding affinity or specificity.

2. Back-mutation Requirements: To preserve the functional conformation of the CDRs and maintain high-affinity binding, it is often necessary to introduce back-mutations in the human framework to match the original murine framework. This is because the interactions between the framework and CDRs are complex and not fully understood, making it challenging to predict which mutations are necessary to retain function.

3. Vernier Zone Residues: Certain residues in the framework, known as vernier zone residues, can influence the conformation of the CDR loops and, consequently, the antibody's affinity and specificity. If these residues are not adequately matched during CDR grafting, the antibody's function may be compromised.

4. Affinity and Stability: CDR grafting can sometimes result in reduced binding affinity and stability. Additional engineering, such as structure-based or library-based methods, is often required to recover or enhance the binding properties of the grafted antibody.

Overall, while CDR grafting is a valuable technique for reducing the immunogenicity of therapeutic antibodies, it does not guarantee that the antibody will retain its original function without further modifications or optimizations. Nor does it guarantee that an antibody will even be expressed in a given system (e.g., CHO or HEK293 cells), as was the case when the 6 Chothia CDRs of chP3R99 were grafted onto the same human variable region framework that was used successfully for IMN12 and IMN26.

### Example 2: Testing Against GAG Microarrays

### Methods

Antibody binding to GAG microarrays was performed by Z Biotech, Inc. (Aurora, CO). Briefly, chP3 and chP3R99 were precomplexed with anti-human IgG (Fc) Cy3 at a ratio of 1:10 (sample:detection antibody) in glycan array assay buffer (GAAB). IMN12 and IMN26 were precomplexed with anti-6×His antibody and anti-mouse IgG (H+L) AF555 in GAAB at a ratio of 10 μg/mL, 1:2500 dilution of the anti-6×His antibody, and 0.4 μg/mL anti-mouse IgG (H+L) AF555. The samples were allowed to precomplex for one hour before serial dilution and application to the array.

The arrays were blocked using glycan array blocking buffer (GABB) for one hour. After the blocking treatment the arrays were washed three times with GAAB, and the samples were applied to the arrays. The samples incubated on the array for one hour at room temperature. After one hour, the array was washed again and then scanned for analysis.

### Results

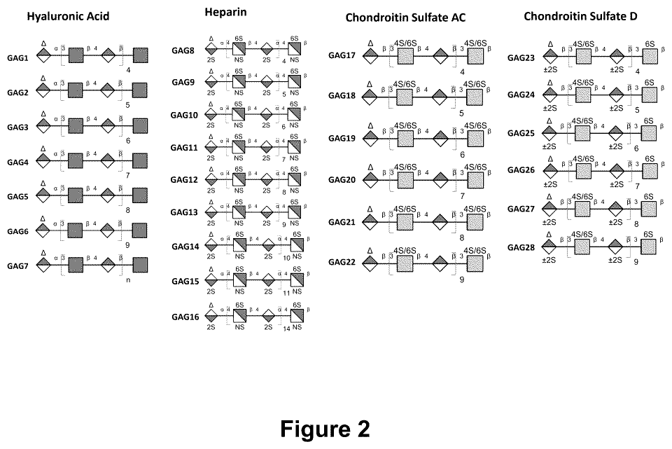

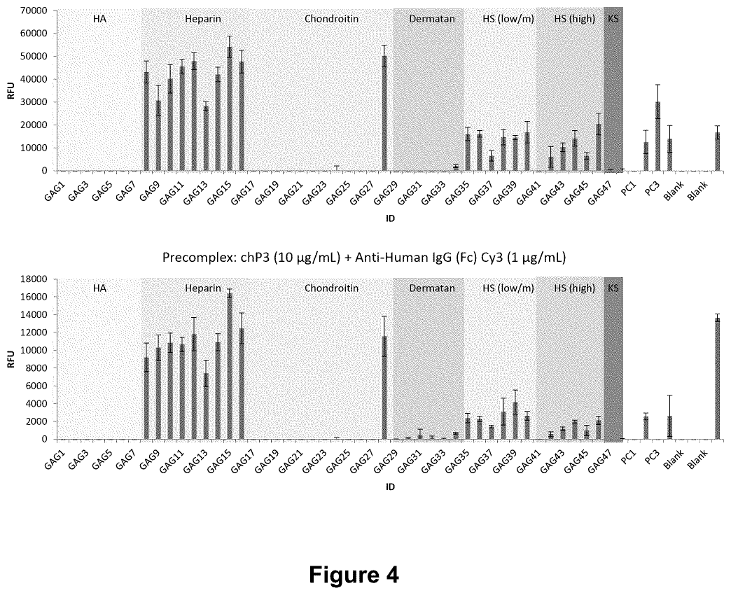

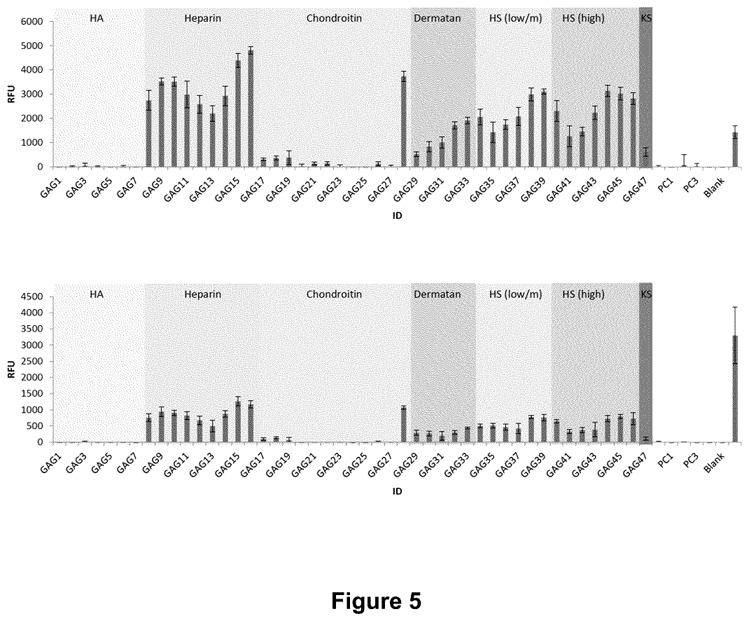

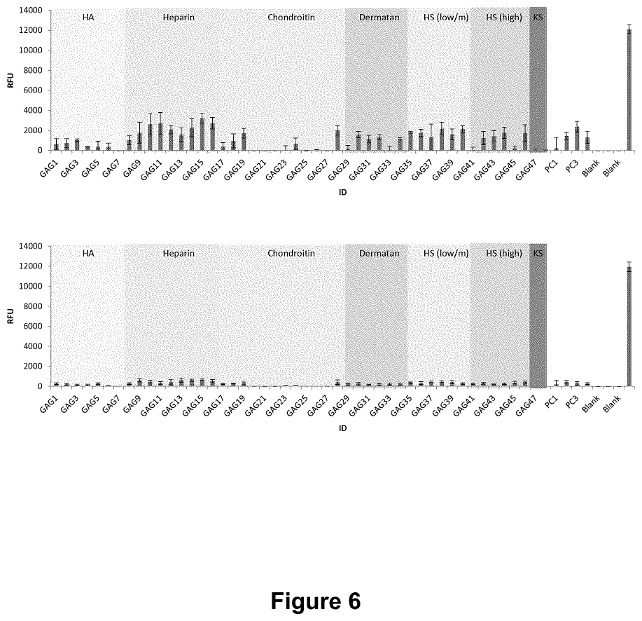

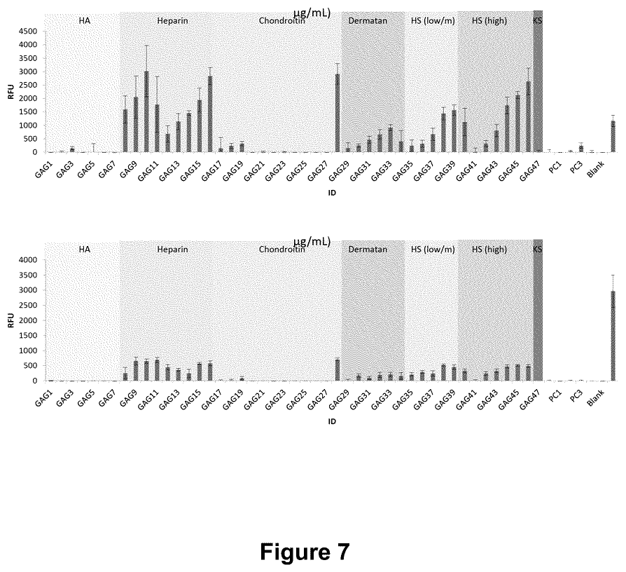

The layout of the GAG microarray is depicted in FIGS. 2 and 3. The GAG microarray results for chP3, IMN12, chP3R99, and IMN26 are depicted in FIGS. 4, 5, 6, and 7, respectively. All four antibodies bound to Hep/HS and long-chain CSD. Additionally, antibodies IMN12, chP3R99, and IMN26 all bound to DS, whereas antibody chP3 did not.

Interestingly, none of the four antibodies tested bound to keratan sulfate (KS), which is the only GAG that contains galactose. Galactose is also found in gangliosides GcGM3, GcGM2, sulfo-asialo-GM1, and SM4, all of which bind to antibody P3 (Vázquez, et al. 1995). Furthermore, the galactose in KS is sulfated, just as the galactose in sulfo-asialo-GM1 and SM4 is sulfated. The difference, however, is that the galactose in KS is sulfated at position 6, whereas the galactose in sulfo-asialo-GM1 and SM4 is sulfated at position 3. Thus, it is evident that antibody P3 can recognize either Neu5Gc or a sulfate group that is attached at position 3 on a galactose molecule, but not a Neu5Gc or a sulfate group that is attached at position 6 on a galactose molecule.

The apparent K_{d} of IMN12 (MW 27.59 kDa), IMN26 (MW 27.32 kDa), and chP3R99 (MW 145.94 kDa) for GAG32 (DS, dp16) was calculated in silico using MotifFinder software (Klamer and Haab, 2021) as follows: 3.28 μg/mL or 118.9 nM for IMN12; 9.64 μg/mL or 352.9 nM for IMN26; and 50.17 μg/mL or 343.8 nM for chP3R99. These results are summarized in Table 1 below. Antibodies IMN26 and ChP3R99 appear to have around the same affinity for DS dp16. Antibody IMN12 appears to have greater affinity for DS dp16 than either IMN26 or chP3R99 on the basis of its lower apparent K_{d}.

K_{d}

| GAG | IMN12 | IMN26 | chP3R99 |
|---|---|---|---|
| GAG32 (DS, dp16) | 118.9 nM | 352.9 nM | 343.8 nM |

### Example 3: Testing Against GSL Glycan Microarrays

### Methods

Antibody binding to glycosphingolipid (GSL) glycan microarrays was performed by Z Biotech, Inc, using microarrays that did not contain the glycosphingolipids themselves, but only the glycan portions thereof. The methodology was essentially the same as for the GAG microarray testing. Briefly, chP3R99 and chP3 were precomplexed with anti-human IgG (Fc) Cy3 at a ratio of 1:10 (sample:detection antibody) in glycan array assay buffer (GAAB). IMN12 and IMN26 were precomplexed with the anti-6×His antibody and anti-mouse IgG (H+L) AF555 in GAAB at a ratio of 10 μg/mL, 1:2500 dilution of the anti-6×His antibody, and 0.4 μg/mL anti-mouse IgG (H+L) AF555. The samples were allowed to precomplex for one hour before serial dilution and application to the array.

The arrays were blocked using glycan array blocking buffer (GABB) for one hour. After the blocking treatment the arrays were washed three times with GAAB, and the samples were applied to the arrays. The samples incubated on the array for one hour at room temperature. After one hour, the array was washed again and then scanned for analysis.

After the assay, the arrays were scanned at 532 nm using high laser intensity (1 PMT). Subsequently, the arrays were analyzed using Mapix (Innopsys, Carbonne, France) microarray analysis software.

### Results

As expected, the markers (streptavidin Cy3 and Cy5), P02 (human IgG), and PC3 (mouse IgG,) showed signals. The layout of the GSL glycan microarray is given below:

| | | | Abbrevation |
|---|---|---|---|
| Type | ID | Glycan Structure | Name |
| Gangli- | G1 | Neu5Acα2-3Galβ1-4Glc | Ac-GM3 |
| oside | G2 | Neu5Gcα2-3Galβ1-4Glc | Gc-GM3 |
| | G3 | Kdnα2-3Galβ1-4Glc | Kdn-GM3 |
| | G4 | Neu5Ac8Meα2-3Galβ1-4Glc | Ac8Me-GM3 |
| | G5 | Neu5Acα2-3(GalNAcβ1-4)Galβ1-4Glc | Ac-GM2 |
| | G6 | Neu5Gcα2-3(GalNAcβ1-4)Galβ1-4Glc | Gc-GM2 |
| | G7 | Kdnα2-3(GalNAcβ1-4)Galβ1-4Glc | Kdn-GM2 |
| | G8 | Neu5Acα2-3(Galβ1-3GalNAcβ1-4)Galβ1-4Glc | Ac-GM1 |
| | G9 | Neu5Gcα2-3(Galβ1-3GalNAcβ1-4)Galβ1-4Glc | Gc-GM1 |
| | G10 | Kdnα2-3(Galβ1-3GalNAcβ1-4)Galβ1-4Glc | Kdn-GM1 |
| | G11 | Neu5Acα2-8Neu5Acα2-3Galβ1-4Glc | Ac-Ac-GD3 |
| | G12 | Neu5Acα2-8Neu5Gcα2-3Galβ1-4Glc | Ac-Gc-GD3 |
| | G13 | Neu5Acα2-8Kdnα2-3Galβ1-4Glc | Ac-Kdn-GD3 |
| | G14 | Neu5Gcα2-8Neu5Acα2-3Galβ1-4Glc | Gc-Ac-GD3 |
| | G15 | Neu5Gcα2-8Neu5Gcα2-3Galβ1-4Glc | Gc-Gc-GD3 |
| | G16 | Kdnα2-8Neu5Acα2-3Galβ1-4Glc | Kdn-Ac-GD3 |
| | G17 | Kdnα2-8Neu5Gcα2-3Galβ1-4Glc | Kdn-Gc-GD3 |
| | G18 | Kdnα2-8Kdnα2-3Galβ1-4Glc | Kdn-Kdn-GD3 |
| | G19 | Neu5Acα2-8Neu5Acα2-3(GalNAcβ1-4)Galβ1-4Glc | Ac-Ac-GD2 |
| | G20 | Neu5Acα2-8Neu5Gcα2-3(GalNAcβ1-4)Galβ1-4Glc | Ac-Gc-GD2 |
| | G21 | Neu5Gcα2-8Neu5Acα2-3(GalNAcβ1-4)Galβ1-4Glc | Gc-Ac-GD2 |
| | G22 | Neu5Gcα2-8Neu5Gcα2-3(GalNAcβ1-4)Galβ1-4Glc | Gc-Gc-GD2 |
| | G23 | Kdnα2-8Neu5Acα2-3(GalNAcβ1-4)Galβ1-4Glc | Kdn-Ac-GD2 |
| | G24 | Kdnα2-8Neu5Gcα2-3(GalNAcβ1-4)Galβ1-4Glc | Kdn-Gc-GD2 |
| | G25 | Kdnα2-8Kdnα2-3(GalNAcβ1-4)Galβ1-4Glc | Kdn-Kdn-GD2 |
| | G26 | Neu5Acα2-3Galβ1-3GalNAcb1-4(Neu5Aca2-3)Galβ1-4Glc | Ac-Ac-GD1a |
| | G27 | Neu5Acα2-3Neu5Acα2-3(Galβ1-3GalNAcβ1-4)Galβ1-4Glc | Ac-Ac-GD1b |
| | G28 | Neu5Gcα2-8Neu5Gcα2-3(Galβ1-3GalNAcβ1-4)Galβ1-4Glc | Gc-Gc-GD1b |
| | G29 | Kdnα2-8Neu5Gcα2-3(Galβ1-3GalNAcβ1-4)Galβ1-4Glc | Kdn-Gc-GD1b |
| | G30 | Neu5Acα2-8Neu5Acα2-3Galβ1-3GalNAcβ1-4(Neu5Aca2- | Ac-Ac-Ac-GT1a |
| | | 3)Galβ1-4Glc | |
| | G31 | GalNAcβ1-4(Neu5Acα2-8Neu5Acα2-8Neu5Acα2-3)Galβ1- | Ac-Ac-Ac-GT2 |
| | | 4Glc | |
| | G32 | Neu5Acα2-8Neu5Acα2-8Neu5Acα2-3Galβ1-4Glc | Ac-Ac-Ac-GT3 |
| Lacto- | G33 | GlcNAcβ1-3Galβ1-4Glc | Lc₃ |
| and | G34 | Galβ1-3GlcNAcβ1-3Galβ1-4Glc | Lc₄ (LNT) |
| Neolacto- | G35 | Galβ1-4GlcNAcβ1-3Galβ1-4Glc | nLc₄ (LNnT) |
| series | G36 | Galβ1-4(Fucα1-3)GlcNAcβ1-3Galβ1-4Glc | Fuc-nLc₄ |
| | G37 | Neu5Acα2-3Galβ1-4GlcNAcβ1-3Galβ1-4Glc | Ac-nLc₄ |
| | G38 | Neu5Gcα2-3Galβ1-4GlcNAcβ1-3Galβ1-4Glc | Gc-nLc₄ |
| | G39 | Kdnα2-3Galβ1-4GlcNAcβ1-3Galβ1-4Glc | Kdn-nLc₄ |
| | G40 | Neu5Ac8Meα2-3Galβ1-4GlcNAcβ1-3Galβ1-4Glc | 8MeAc-nLc₄ |
| | G41 | Neu5Acα2-3Galβ1-3GlcNAcβ1-3Galβ1-4Glc | Ac-Lc₄ |
| | G42 | Neu5Gcα2-3Galβ1-3GlcNAcβ1-3Galβ1-4Glc | Gc-Lc₄ |
| | G43 | Kdnα2-3Galβ1-4GlcNAcβ1-3Galβ1-4Glc | Kdn-Lc₄ |
| | G44 | Neu5Ac8Meα2-3Galβ1-3GlcNAcβ1-3Galβ1-4Glc | 8MeAc-Lc₄ |
| | G45 | Neu5Gcα2-3Galβ1-4(Fucα1-3)GlcNAcβ1-3Galβ1-4Glc | Gc-Fuc-nLc₄ |
| | G46 | Kdnα2-3Galβ1-4(Fucα1-3)GlcNAcβ1-3Galβ1-4Glc | Kdn-Fuc-nLc₄ |
| Globo- | G47 | Galα1-4Galβ1-4Glc | Gb₃ |
| and | G48 | Galα1-3Galβ1-4Glc | iGb₃ |
| Isoglobo- | G49 | GalNAcβ1-3Galα1-4Galβ1-4Glc | Gb₄ |
| series | G50 | GalNAcβ1-3Galα1-3Galβ1-4Glc | iGb₄ |
| | G51 | Galβ1-3GalNAcβ1-3Galα1-4Galβ1-4Glc | Gb₅ (SSEA-3) |
| | G52 | Galβ1-3GalNAcβ1-3Galα1-3Galβ1-4Glc | iGb₅ |
| | G53 | Fucα1-2Galβ1-3GalNAcβ1-3Galα1-4Galβ1-4Glc | Globo-H |
| | G54 | Neu5Gcα2-3Galβ1-3GalNAcβ1-3Galα1-4Galβ1-4Glc | Gc-Gb₅ |
| | G55 | Kdnα2-3Galβ1-3GalNAcβ1-3Galα1-4Galβ1-4Glc | Kdn-Gb₅ |
| | G56 | Neu5Acα2-3Galβ1-3GalNAcβ1-3Galα1-3Galβ1-4Glc | Ac-iGb₅ |
| | G57 | Neu5Gcα2-3Galβ1-3GalNAcβ1-3Galα1-3Galβ1-4Glc | Gc-iGb₅ |
| | G58 | Kdnα2-3Galβ1-3GalNAcβ1-3Galα1-3Galβ1-4Glc | Kdn-iGb₅ |

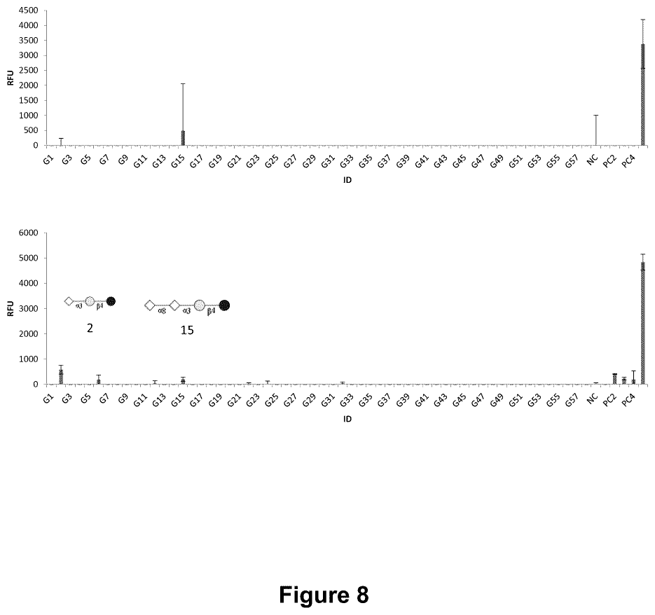

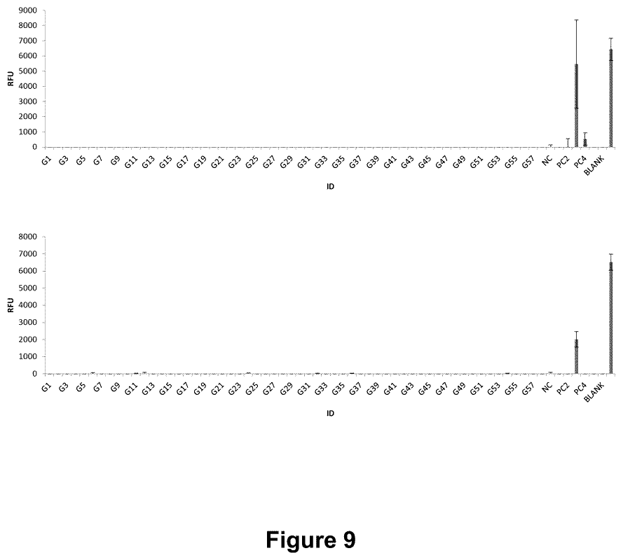

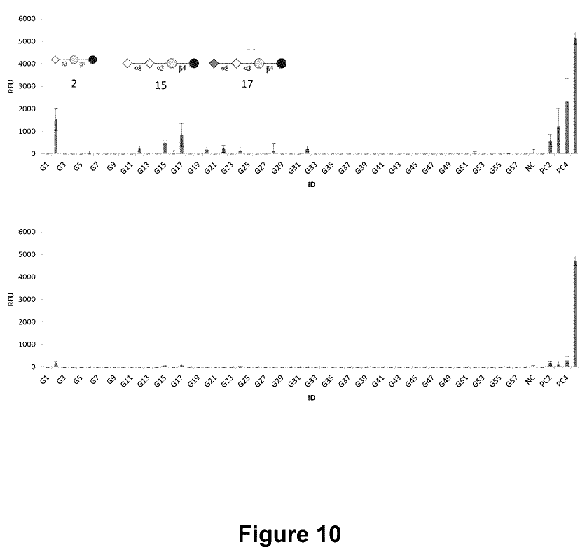

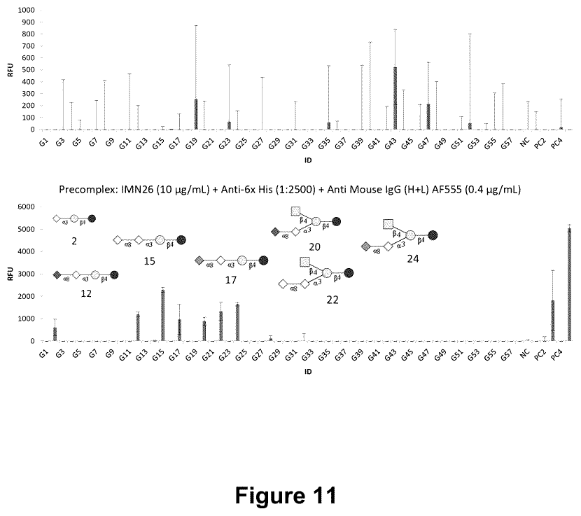

The GSL glycan microarray results for chP3, IMN12, chP3R99, and IMN26 are depicted in FIGS. 8, 9, 10, and 11, respectively.

GSL glycan microarray testing revealed that antibody chP3 bound to GcGM3 and GcGcGD3. Antibody IMN12 failed to bind to any of the GSL glycans on the microarray. Antibody chP3R99 bound to GcGM3, GcGcGD3, and KdnGcGD3. In contrast, antibody IMN26 bound to GcGM3, multiple types of GcGD3 structure, and multiple types of GcGD2 structure; basically, every structure tested with a Neu5Gc linked α2-3 to the core galactose.

Antibody IMN12 has Kabat CDRs that are identical to the Kabat CDRs of chP3R99, and it has IMGT CDRs that are identical to the IMGT CDRs of chP3R99 except for VHCDR1 (SEQ ID NO:14). Yet chP3R99 binds to Neu5Gc-containing gangliosides whereas IMN12 does not. The IMGT VHCDR1 of chP3R99 must therefore play a critical role in chP3R99 binding to Neu5Gc-containing gangliosides.

Taken together with the GAG microarray results presented in Example 2, the present GSL glycan microarray results indicate that antibody IMN26 replicates both the anti-GAG and anti-Neu5Gc functions of chP3R99, whereas antibody IMN12 does not. Antibody IMN12 shares the same 6 Kabat CDRS as chP3R99, but not the same 6 IMGT CDRs. Antibody IMN26 shares the same 6 IMGT CDRs as chP3R99, but not the same 6 Kabat CDRs.

Antibodies IMN12 and IMN26 each represent a significant improvement over antibodies P3, chP3, chP3R99, and chP3R99-LALA by virtue of the fully human amino acid sequences of their variable region frameworks. Antibodies P3, chP3, chP3R99, and chP3R99-LALA could each potentially elicit a human anti-mouse antibody (HAMA) response. The HAMA response is a significant immunological reaction that occurs when human patients are exposed to mouse-derived proteins, particularly mAbs. This response can have various clinical implications, especially in the context of diagnostic and therapeutic applications.

Since the variable region frameworks of antibodies IMN12 and IMN26 comprise fully human amino acid sequences, neither antibody will elicit a HAMA response. Antibodies IMN12 and IMN26 are therefore more immunologically compatible with humans than antibodies P3, chP3, chP3R99, or chP3R99-LALA.

Antibodies IMN12 and IMN26 are also functionally distinct from antibodies P3 and chP3R99, even though antibody IMN12 contains the same Kabat CDRs as antibody chP3R99, and antibody IMN26 contains the same IMGT CDRs as antibody chP3R99. Both antibodies IMN12 and IMN26 have a pattern of reactivity against GAGs and GSL glycans that differs from the pattern of reactivity of chP3 and chP3R99.

What antibodies IMN12, IMN26, and chP3R99 all have in common is a similar pattern of reactivity against GAGs, including DS. When administered intravenously and on an acute basis to insulin resistant rats, chP3R99 has the ability to block LDL and remnant lipoprotein binding in vivo to the extracellular matrix of the arterial endothelium, which is rich in DS (Soto, et al. 2024). It would therefore be expected that antibodies IMN12 and IMN26 would have a similar effect on LDL and remnant lipoprotein binding to the arterial endothelium when administered on an acute basis, since both antibodies have similar, or (in the case of IMN12) even better affinity for DS, than chP3R99.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Borén J, Chapman M J, Krauss R M, Packard C J, Bentzon J F, Binder C J, Daemen M J, Demer L L, Hegele R A, et al. Low-density lipoproteins cause atherosclerotic cardiovascular disease: pathophysiological, genetic, and therapeutic insights: a consensus statement from the European Atherosclerosis Society Consensus Panel. Eur Heart J 41:2313-2330, 2020.
Brito V, Mellal K, Portelance S G, Pérez A, Soto Y, deBlois D, Ong H, Marleau S, and Vázquez. A M. Induction of anti-anti-idiotype antibodies against sulfated glycosaminoglycans reduces atherosclerosis in alipoprotein E-deficient mice. Arterioscler Thromb Vasc Biol 32:2847-2854, 2012.
Brito V, Mellal, Zoccal K F, Soto Y, Menard L, Sarduy R, Faccioli L H, Ong H, Vázquez. A M, and Marleau S. Atheroregressive potential of the treatment with a chimeric monoclonal antibody against sulfated glycosaminoglycans on pre-existing lesions in apolipoprotein E-deficient mice. Front Pharmacol 8:782, 2017.
Cardin A D, and Weintraub H J. Molecular modeling of protein-glycosaminoglycan interactions. Arteriosclerosis 9(1):21-32, 1989.
Chan A, Torelli S, Cheng E, Batchelder R, Waliany S, Neal J, Witteles R, Nguyen P, Cheng P, and Zhu H. Immunotherapy-associated atherosclerosis: a comprehensive review of recent findings and implications for future research. Curr Treat Options Cardio Med 25:715-735, 2023.
Dhar C, Sasmal A, and Varki A. From “serum sickness” to “xenosialitis”: past, present, and future significance of the non-human sialic acid Neu5Gc. Front Immunol 10:807, 2019.
Delgado-Roche L, Acosta E, Soto Y, Hernández-Matos Y, Olivera A, Fernández-Sanchez E, and Vázquez A M. The treatment with an anti-glycosaminoglycan antibody reduces aortic oxidative stress in a rabbit model of atherosclerosis. Free Radical Res 47(4):309-315, 2013.
Delgado-Roche L, Brito V, Acosta E, Pérez A, Fernández JR, Hernández-Matos Y, Griñan T, Soto Y, León OS, Marleau S, and Vázquez A M. Arresting progressive atherosclerosis by immunization with an anti-glycosaminoglycan monoclonal antibody in apolipoprotein E-deficient mice. Free Radical Biol Med 89:557-566, 2015.
Fernández-Marrero Y, Hernández T, Roque-Navarro L, Talavera A, Moreno E, Griñan T, Vásquez A M, Mateo de Acosta C, Pérez R, and López-Requena A. Switching on cytotoxicity by a single mutation at the heavy chain variable region of an anti-ganglioside antibody. Mol Immunol 48:1059-1067, 2011.
Jiang H, Zhou Y, Nabavi S M, Sahebkar A, Little P J, Xu S, Weng J, and Ge J. Mechanisms of oxidized LDL-mediated endothelial dysfunction and its consequences for the development of atherosclerosis. Frontiers Cardiovasc Med 9:925923, 2022.
Khambhati J, Engels M, Allard-Ratick M, Sandesara P B, Quyyumi A A, and Sperling L. Immunotherapy for the prevention of atherosclerotic cardiovascular disease: promise and possibilities. Atherosclerosis 276:1-9, 2018.
Klamer Z and Haab B. Combined analysis of multiple glycan-array datasets: new explorations of protein-glycan interactions. Anal Chem 93(31):10925-10933, 2021.
Labrada M, Dorvignit D, Hevia G, Rodriguez-Zhurbenko N, Hernández A M, Vázquez A M, and Fernández LE. GM3(Neu5Gc) ganglioside: an evolution fixed neoantigen for cancer immunotherapy. Seminars in Oncology 45:41-51, 2018.
López-Requena A, Mateo de Acosta C, Moreno E, Gonzalez M, Puchades Y, Talavera A, Vispo N S, Vázquez A M, and Pérez R. Gangliosides, Ab1 and Ab2 antibodies I. Towards a molecular dissection of an idiotype—anti-idiotype system. Mol Immunol 44: 423-433, 2007.
López-Requena A, Mateo de Acosta C, Pérez A, Valle A, Lombardero J, Sosa K, Pérez R, and Vázquez A M. Chimeric anti-N-glycolyl-ganglioside and its anti-idiotypic MAbs: immunodominance of their variable regions. Hybridoma Hybridomics 22(4): 235-243, 2003.
Meng Q, Liu H, Liu J, Pang Y, and Liu Q. Advances in immunotherapy modalities for atherosclerosis. Front Pharmacol 13:1079185.
Moreno E, Lanne B, Vázquez A M, Kawashima I, Tai T, Fernández LE, Karlsson K-A, Angstrom J, and Pérez R. Delineation of the epitope recognized by an antibody specific for N-glycolylneuraminic acid-containing gangliosides. Glycobiology 8(7):695-705, 1998.
Perez A, Lombardero J, Mateo C, Mustelier G, Alfonso M, Vázquez A M, and Perez R. Immunogenetic analysis of variable regions encoding AB1 and γ-type AB2 antibodies from the NeuGc-containing ganglioside family. Hybridoma 20(4):211-221, 2001.
Sarduy R, Brito V, Castillo A, Soto Y, Griñan T, Marleau S, and Vázquez A M. Dose-dependent induction of an anti-idiotypic cascade by anti-glycosaminoglycan monoclonal antibody in apoE^{-/-} mice: association with atheroprotection. Front Immunol 8:232, 2017.
Shah S R, Abbasi Z, Fatima M, Ochani R K, Shahnawaz W, Khan M A, and Shah S A. Canakinumab and cardiovascular outcomes: results of the CANTOS trial. J Comm Hosp Int Med Perspect 8:21-22, 2018.
Soto Y, Acosta E, Delgado L, Pérez A, Falcón V, Bécquer MA, Fraga A, Brito V, Alvarez I, Griñan T, Fernández-Marrero Y, López-Requena A, Noa M, Fernández E, and Vázquez A M. Antiatherosclerotic effect of an antibody that binds to extracellular matrix glycosaminoglycans. Arterioscler Thromb Vasc Biol 32:595-604, 2012.
Soto Y, Hernández A, Sarduy R, Brito V, Marleau S, Vine D F, Vázquez A M, and Proctor S D. Novel chP3R99 mAb reduces subendothelial retention of atherogenic lipoproteins in Insulin-Resistant rats: acute treatment versus long-term protection as an idiotypic vaccine for atherosclerosis. Vaccine Weekly (pre-print). Updated 6 March, 2024.
Soto Y, Mesa N, Alfonso Y, Pérez A, Batlle F, Griñan T, Pino A, Viera J, Frómeta M, Brito V, Olivera A, Zayas F, and Vázquez A. Targeting arterial wall sulfated glycosaminoglycans in rabbit atherosclerosis with a mouse/human chimeric antibody. mAbs 6(5):1340-1346, 2014.
Suero-Abreu G A, Zanni M V, and Neilan T G. Atherosclerosis with immune checkpoint inhibitor therapy. J Am Coll CardioOnc 4(5):598-615, 2022.
Vázquez A M, Alfonso M, Lanne B, Karlsson K-A, Carr A, Barroso O, Fernández LE, Rengifo E, Lanio M E, Alvarez C, Zeuthen J, and Pérez R. Generation of a murine monoclonal antibody specific for N-glycolylneuraminic acid-containing gangliosides that also recognizes sulfated glycolipids. Hybridoma 14(6):551-556, 1995.

SEQUENCES

SEQ ID NO: 1 = P3 VH sequence
QVQLKESGPGLVAPSQSLSITCTVSGFSLSRYSVHWVRQPPGKGLEW
LGMIWGGGSTDYNSALKSRLSISKDNSKSQVFLKMNSLQTDDTAMYY
CARSGVREGRAQAWFAYWGQGTLVTVSA
SEQ ID NO: 2 = P3 VL sequence
DIVMTQSHKFMSTSVGDRVSITCKASQDVSTAVAWYQQKPGQSPKLL
IYSASYRYTGVPDRFTGSGSGTDFTFTISSVQAEDLAVYYCQQHYST
PWTFGGGTKLELK
SEQ ID NO: 3 = P3 Kabat VHCDR1
RYSVH
SEQ ID NO: 4 = P3 Kabat VHCDR2
MIWGGGSTDYNSALKS
SEQ ID NO: 5 = P3 Kabat VHCDR3
SGVREGRAQAWFAY
SEQ ID NO: 6 = P3 Kabat VLCDR1
KASQDVSTAVA
SEQ ID NO: 7 = P3 Kabat VLCDR2
SASYRYT
SEQ ID NO: 8 = P3 Kabat VLCDR3
QQHYSTPWT
SEQ ID NO: 9 = chP3R99 Kabat VHCDR3
SGVRRGRAQAWFAY
SEQ ID NO: 10 = chP3R99 heavy chain (Kabat
CDRs in bold)
QVQLKESGPGLVAPSQSLSITCTVSGFSLSRYSVHWVRQPPGKGLEW
LGMIWGGGSTDYNSALKSRLSISKDNSKSQVFLKMNSLQTDDTAMYY
CARSGVRRGRAQAWFAYWGQGTLVTVSAASTKGPSVFPLAPSSKSTS
GGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCP
APELLGGPSVFLFPPKPKDTLMISRTPEVTCVVVDVSHEDPEVKFNW
YVDGVEVHNAKTKPREEQYNSTYRVVSVLTVLHQDWLNGKEYKCKVS
NKALPAPIEKTISKAKGQPREPQVYTLPPSRDELTKNQVSLTCLVKG
FYPSDIAVEWESNGQPENNYKTTPPVLDSDGSFFLYSKLTVDKSRWQ
QGNVFSCSVMHEALHNHYTQKSLSLSPGK
SEQ ID NO: 11 = chP3R99 light chain (Kabat
CDRs in bold)
DIVMTQSHKFMSTSVGDRVSITCKASQDVSTAVAWYQQKPGQSPKLL
IYSASYRYTGVPDRFTGSGSGTDFTFTISSVQAEDLAVYYCQQHYST
PWTFGGGTKLELKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
SEQ ID NO: 12 = IMN12 (Kabat CDRs in bold)
QVQLVESGGGVVQPGRSLRLSCAASGFPFRRYSVHWVRQALGKGLEW
VAMIWGGGSTDYNSALKSRFTISRDTSKNTVYLKMNRLRTEDTAVYY
CARSGVRRGRAQAWFAYWGKGTTVTVSSGGGGSGGGGSGGGGSDIVL
TQSPGTLSLSPGERATLSCKASQDVSTAVAWYQQKPGQAPRLLIYSA
SYRYTGMPDRFSGSGSGTDFTLTISRLEPEDFAVYYCQQHYSTPWTF
GGGTKVEIKHHHHHH
SEQ ID NO: 13 = IMN26 (IHGT CDRs in bold)
QVQLVESGGGVVQPGRSLRLSCAASGFSLSRYSMHWVRQALGKGLEW
VAVIWGGGSTYYADSVKGRFTISRDTSKNTVYLKMNRLRTEDTAVYY
CARSGVRRGRAQAWFAYWGKGTTVTVSSGGGGSGGGGSGGGGSDIVL
TQSPGTLSLSPGERATLSCRASQDVSTALAWYQQKPGQAPRLLIYSA
STRATGMPDRFSGSGSGTDFTLTISRLEPEDFAVYYCQQHYSTPWTF
GGGTKVEIKHHHHHH
SEQ ID NO: 14 = chP3R99 VH region
QVQLKESGPGLVAPSQSLSITCTVSGFSLSRYSVHWVRQPPGKGLEW
LGMIWGGGSTDYNSALKSRLSISKDNSKSQVFLKMNSLQTDDTAMYY
CARSGVRRGRAQAWFAYWGQGTLVTVSA
SEQ ID NO: 15 = chP3R99, IMN12 and IMN26 IHGT
VHCDR2
IWGGGST
SEQ ID NO: 16 = chP3R99, IMN12 and IMN26 IHGT
VHCDR3
ARSGVRRGRAQAWFAY
SEQ ID NO: 17 = chP3R99, IMN12 and IMN26 IHGT
VLCDR1
QDVSTA
SEQ ID NO: 18 = chP3R99 VL region
DIVMTQSHKFMSTSVGDRVSITCKASQDVSTAVAWYQQKPGQSPKLL
IYSASYRYTGVPDRFTGSGSGTDFTFTISSVQAEDLAVYYCQQHYST
PWTFGGGTKLELK
SEQ ID NO: 19 = chP3R99, IMN12 and IMN26 IHGT
VLCDR3
QQHYSTPWT
SEQ ID NO: 20 = chP3R99 and IMN26 IHGT VHCDR1
GFSLSRYS
SEQ ID NO: 21 = IMN12 IHGT VHCDR1
GFPFRRYS
SEQ ID NO: 22 = IMN12 VH region (Kabat CDRs in
bold)
QVQLVESGGGVVQPGRSLRLSCAASGFPFRRYSVHWVRQALGKGLEW
VAMIWGGGSTDYNSALKSRFTISRDTSKNTVYLKMNRLRTEDTAVYY
CARSGVRRGRAQAWFAYWGKGTTVTVSS
SEQ ID NO: 23 = IMN12 VL region (Kabat CDRs in
bold)
DIVLTQSPGTLSLSPGERATLSCKASQDVSTAVAWYQQKPGQAPRLL
IYSASYRYTGMPDRFSGSGSGTDFTLTISRLEPEDFAVYYCQQHYST
PWTFGGGTKVEIK
SEQ ID NO: 24 = IMN26 VH region (IHGT CDRs in
bold)
QVQLVESGGGVVQPGRSLRLSCAASGFSLSRYSMHWVRQALGKGLEW
VAVIWGGGSTYYADSVKGRFTISRDTSKNTVYLKMNRLRTEDTAVYY
CARSGVRRGRAQAWFAYWGKGTTVTVSS
SEQ ID NO: 25 = IMN26 VL region (IHGT CDRs in
bold)
DIVLTQSPGTLSLSPGERATLSCRASQDVSTALAWYQQKPGQAPRLL
IYSASTRATGMPDRFSGSGSGTDFTLTISRLEPEDFAVYYCQQHYST
PWTFGGGTKVEIK

1. (canceled)

2. (canceled)

3. An antibody which specifically binds to sulfated glycosaminoglycans, wherein said antibody comprises a human heavy chain variable region sequence of SEQ ID NO: 22 or 24; a human light chain variable region sequence of SEQ ID NO: 23 or 25; or both a human heavy chain variable region sequence of SEQ ID NO: 22 or 24 and a human light chain variable region sequence of SEQ ID NO: 23 or 25.

4. The antibody of claim 3, wherein said antibody comprises a human heavy chain variable region sequence of SEQ ID NO: 22 and a human light chain variable region sequence of SEQ ID NO: 23; or wherein said antibody comprises a human heavy chain variable region sequence of SEQ ID NO: 24 and a human light chain variable region sequence of SEQ ID NO: 25.

5. (canceled)

6. An antibody which specifically binds to sulfated glycosaminoglycans, wherein said antibody comprises an amino acid sequence of SEQ ID NO: 12 or 13.

7. The antibody of claim 3, wherein the antibody is a monoclonal antibody.

8. The antibody of claim 3, wherein the antibody does not bind to gangliosides containing N-glycolylneuraminic acid linked α2-3 to a core galactose.

9. The antibody of claim 3, wherein the antibody binds to gangliosides containing N-glycolylneuraminic acid linked α2-3 to a core galactose.

10. The antibody of claim 6, wherein the antibody is a single-chain Fv monoclonal antibody which specifically binds to gangliosides containing N-glycolylneuraminic acid linked α2-3 to a core galactose and/or sulfated glycosaminoglycans, wherein said single-chain Fv monoclonal antibody comprises the amino acid sequence of SEQ ID NO:12 or 13.

11. The single-chain Fv monoclonal antibody of claim 10 which specifically binds to sulfated glycosaminoglycans and to gangliosides containing N-glycolylneuraminic acid linked α2-3 to the core galactose.

12. The single-chain Fv monoclonal antibody of claim 11, comprising the amino acid sequence of SEQ ID NO:13.

13. The single-chain Fv monoclonal antibody of claim 10 which does not bind to gangliosides containing N-glycolylneuraminic acid linked α2-3 to the core galactose.

14. The single-chain Fv monoclonal antibody of claim 13, comprising the amino acid sequence of SEQ ID NO:12.

15. The monoclonal antibody of claim 7 which specifically binds to gangliosides containing N-glycolylneuraminic acid linked α2-3 to a core galactose and/or sulfated glycosaminoglycans, wherein said monoclonal antibody comprises a heavy chain variable region comprising the amino acid sequence of SEQ ID NO: 22 or 24 and a light chain variable region comprising the amino acid sequence of SEQ ID NO: 23 or 25.

16. The monoclonal antibody of claim 15, which specifically binds to sulfated glycosaminoglycans and to gangliosides containing N-glycolylneuraminic acid linked α2-3 to the core galactose.

17. The monoclonal antibody of claim 16, comprising the amino acid sequence of SEQ ID NO:13.

18. A method of reducing and/or preventing binding of low density lipoprotein (LDL) to the extracellular matrix of the arterial endothelium in a subject in need thereof, or to blood vessels in a subject in need thereof, the method comprising administering the antibody of claim 3 to the subject.

19. (canceled)

20. The method of claim 18, for reducing and/or preventing binding of LDL to the endothelial surface of blood vessels.

21. The method of claim 18, wherein the blood vessels comprise atherosclerotic arteries.

22. A method of diagnosing atherosclerosis in a subject, or of imaging atherosclerosis in a subject, the method comprising administering the antibody of claim 3 to the subject, and visualizing the antibody in the subject.

23. (canceled)

24. The method of claim 22, wherein the antibody comprises a tag for visualising and localizing the antibody in the subject.

25. The method of claim 22, wherein the subject is a mammal or a human.

26. (canceled)
